(19) United States
(12) Patent Application Publication
(10) Pub. No.: US 2022/0409912 A1
(43) Pub. Date: Dec. 29, 2022

(54) ELECTRIC DEVICE FOR DEFIBRILLATION, AND METHOD FOR GENERATING DEFIBRILLATION SIGNAL

(71) Applicant: KANEKA CORPORATION, Osaka (JP)

(72) Inventor: Shinichiro SAKAMOTO, Settsu-shi (JP)

(73) Assignee: KANEKA CORPORATION, Osaka (JP)

(21) Appl. No.: 17/900,947

(22) Filed: Sep. 1, 2022

Related U.S. Application Data
(63) Continuation of application No. PCT/JP2021/002247, filed on Jan. 22, 2021.

(30) Foreign Application Priority Data
Mar. 9, 2020 (JP) 2020-040144

(51) Int. Cl. A61N 1/39; A61B 5/00; A61B 5/352

(52) U.S. Cl. CPC A61N 1/3987; A61B 5/7239; A61B 5/352

(57) ABSTRACT

An object of the present invention is to provide a new electric device for defibrillation and a method for generating a defibrillation signal. The electric device for defibrillation includes an electrocardiogram waveform input unit; and an enable signal generating unit, wherein the electric device for defibrillation is configured to generate an enable signal from the enable signal generating unit after a peak of an event is surpassed and when or after condition 1 is satisfied, the event being estimated to be an R-wave of an electrocardiogram waveform, the electrocardiogram waveform being obtained from a human body and inputted from the electrocardiogram waveform input unit, and the condition 1 is that a differential value in a differentiated waveform generated based on the electrocardiogram waveform, which corresponds to the event estimated to be the R-wave, is a negative constant C₃ value or less.

### TECHNICAL FIELD

The present invention relates to an electric device for defibrillation and a method for generating a defibrillation signal.

### BACKGROUND ART

In treatments for arrhythmia such as atrial fibrillation and ventricular fibrillation, defibrillation in which a heart rhythm is brought back to a normal rhythm by delivering an electrical stimulus is performed. Defibrillation is performed with an automated external defibrillator (AED), an implantable cardioverter defibrillator (ICD), a defibrillation paddle system, a defibrillation catheter system, etc.

As an example of such a defibrillation catheter system, Patent Document 1 discloses a system including input means for receiving an ECG waveform; processing means for processing the ECG waveform based on a probability density function, thereby forming an output signal; a heart rate detection device; and processing output means, and when the processing output means receives a predetermined signal from at least one of the processing means and the heart rate detection device, the processing means and the heart rate detection device are connected to a defibrillation pulse generator so as to start defibrillation shock delivery. Furthermore, Patent Document 1 describes that the heart rate detection device includes wave detecting means, and the wave detecting means includes comparing means for differentiating an ECG signal and extracting an absolute value of the differentiated signal, thereby obtaining a slew rate, and providing a slew rate output signal when the slew rate exceeds a predetermined slew rate threshold.

### RELATED ART DOCUMENTS

### Patent Document

Patent Document 1: JP-T-59-500895

### SUMMARY OF THE INVENTION

### Problems to be Solved by the Invention

Conventionally, in treatments for atrial fibrillation, defibrillation needs to be performed during an absolute refractory period during which the ventricles contract and even if a stimulus is applied, the ventricles do not respond. If a stimulus is delivered to the heart during a period other than the absolute refractory period, then ventricular fibrillation may occur. Hence, in a conventional defibrillation catheter system, a voltage needs to be applied in synchronization with an R-wave which is a waveform obtained upon ventricular contraction. Specifically, an electric device for defibrillation in the conventional defibrillation catheter system generates an enable signal for application of a voltage for defibrillation during a period from a rise of an R-wave to a peak of the R-wave, but there is a possibility that a T-wave may be erroneously recognized as an R-wave and a voltage for defibrillation may be applied, causing ventricular fibrillation. Hence, in recent years, there has been a demand for developing an electric device for defibrillation that includes a new mechanism for generating an enable signal.

The present invention is made in view of the above-described circumstances, and an object of the present invention is to provide a new electric device for defibrillation and a method for generating a defibrillation signal.

### Solutions to the Problems

An electric device for defibrillation according to the present invention that can solve the above-described problem is as follows:

[1] An electric device for defibrillation including:

an electrocardiogram waveform input unit; and

an enable signal generating unit, wherein

the electric device for defibrillation is controlled to generate an enable signal from the enable signal generating unit after a peak of an event is surpassed and when or after condition 1 is satisfied, the event being estimated to be an R-wave of an electrocardiogram waveform, the electrocardiogram waveform being obtained from a human body and inputted from the electrocardiogram waveform input unit, the condition 1 being that a differential value generated from the event estimated to be the R-wave is a negative constant C₃ value or less.

In the electric device for defibrillation according to the present invention, as described above, a threshold value (negative constant C₃ value) is provided for a differential value of a portion of an R-wave of an electrocardiogram waveform that corresponds to a fall phase occurring after a peak of the R-wave is surpassed, and an electric device for defibrillation having this configuration does not exist conventionally. Furthermore, by having this configuration, only an R-wave that generally has an abrupt fall can be accurately detected, making it easier to determine whether an application-target waveform is an R-wave, and thus, application of a voltage associated with erroneous detection of an R-wave can be easily avoided.

Furthermore, preferred aspects of the electric device for defibrillation and a method for generating a defibrillation signal of the present invention are as shown in the following [2] to [17]:

[2] The electric device for defibrillation according to above [1], wherein the electric device for defibrillation is controlled to generate the enable signal when or after condition 2 is satisfied and the condition 1 is satisfied, the condition 2 being that a peak value of a differentiated waveform (hereinafter, simply referred to as “positive wave”) is a positive constant C₁ value or more, the differentiated waveform being a set of differential values generated from a portion of the event estimated to be the R-wave that corresponds to a rise phase occurring before the peak of the event estimated to be the R-wave.

[3] The electric device for defibrillation according to above [2], wherein the electric device for defibrillation is controlled to generate the enable signal when or after the condition 2 and condition 3 are satisfied and the condition 1 is satisfied, the condition 3 being that a period of time during which the differential values of the positive wave are a positive constant C₂ value or more is measured and is 10 milliseconds or more and 80 milliseconds or less, the positive constant C₂ value being smaller than the C₁ value.

[4] The electric device for defibrillation according to any one of above [1] to [3], wherein the electric device for defibrillation is controlled to generate the enable signal when or after condition 4 is satisfied and the condition 1 is satisfied, the condition 4 being that a period of time from when a differential value generated from an event estimated to be an R-wave (hereinafter, simply referred to as “Rₙ₋₁-wave”) immediately before the event estimated to be the R-wave (hereinafter, simply referred to as “Rₙ-wave”) reaches the C₃ value until the differential value generated from the Rₙ-wave reaches the C₃ value is measured and is 50 milliseconds or more.

[5] The electric device for defibrillation according to any one of above [1] to [4], wherein a portion from the electrocardiogram waveform input unit to the enable signal generating unit is formed of a hardware circuit.

[6] The electric device for defibrillation according to any one of above [1] to [5], including a display unit that displays the electrocardiogram waveform, wherein

the electric device for defibrillation is controlled to generate a mark display signal for providing a mark to an event estimated to be an R-wave on the display unit from a mark display signal generating unit after a peak of the event estimated to be the R-wave is surpassed and when or after condition 1 is satisfied, the condition 1 being that a differential value generated from the event estimated to be the R-wave is a negative constant C₃ value or less.

[7] The electric device for defibrillation according to above [6], wherein the electric device for defibrillation is controlled to generate the mark display signal when or after condition 2 is satisfied and the condition 1 is satisfied, the condition 2 being that a peak value of a differentiated waveform (hereinafter, simply referred to as “positive wave”) is a positive constant C₁ value or more, the differentiated waveform being a set of differential values generated from a portion of the event estimated to be the R-wave that corresponds to a rise phase occurring before the peak of the event estimated to be the R-wave.

[8] The electric device for defibrillation according to above [7], wherein the electric device for defibrillation is controlled to generate the mark display signal when or after the condition 2 and condition 3 are satisfied and the condition 1 is satisfied, the condition 3 being that a period of time during which the differential values of the positive wave are a positive constant C₂ value or more is measured and is 10 milliseconds or more and 80 milliseconds or less, the positive constant C₂ value being smaller than the C₁ value.

[9] The electric device for defibrillation according to any one of above [6] to [8], wherein the electric device for defibrillation is controlled to generate the mark display signal when or after condition 4 is satisfied and the condition 1 is satisfied, the condition 4 being that a period of time from when a differential value generated from an event estimated to be an R-wave (hereinafter, simply referred to as “Rₙ₋₁-wave”) immediately before the event estimated to be the R-wave (hereinafter, simply referred to as “Rₙ-wave”) reaches the C₃ value until the differential value generated from the Rₙ-wave reaches the C₃ value is measured and is 50 milliseconds or more.

[10] A method for generating a defibrillation signal, the method including the steps of:

determining whether condition 1 is satisfied after a peak of an event is surpassed, the event being estimated to be an R-wave of an electrocardiogram waveform obtained from a human body, the condition 1 being that a differential value generated from the event estimated to be the R-wave is a negative constant C₃ value or less; and

generating an enable signal when or after the condition 1 is satisfied.

[11] The method for generating a defibrillation signal according to above [10], further including the step of determining whether condition 2 is satisfied, the condition 2 being that a peak value of a differentiated waveform (hereinafter, simply referred to as “positive wave”) is a positive constant C₁ value or more, the differentiated waveform being a set of differential values generated from a portion of the event estimated to be the R-wave that corresponds to a rise phase occurring before the peak of the event estimated to be the R-wave.

[12] The method for generating a defibrillation signal according to above [11], further including the step of determining whether condition 3 is satisfied, the condition 3 being that a period of time during which the differential values of the positive wave are a positive constant C₂ value or more is measured and is 10 milliseconds or more and 80 milliseconds or less, the positive constant C₂ value being smaller than the C₁ value.

[13] The method for generating a defibrillation signal according to any one of above [10] to [12], further including the step of determining whether condition 4 is satisfied, the condition 4 being that a period of time from when a differential value generated from an event estimated to be an R-wave (hereinafter, simply referred to as “Rₙ₋₁-wave”) immediately before the event estimated to be the R-wave (hereinafter, simply referred to as “Rₙ-wave”) reaches the C₃ value until the differential value generated from the Rₙ-wave reaches the C₃ value is measured and is 50 milliseconds or more.

[14] The method for generating a defibrillation signal according to any one of above [10] to [13], including the steps of:

determining whether condition 1 is satisfied after a peak of an event is surpassed, the event being estimated to be an R-wave of an electrocardiogram waveform obtained from a human body, the condition 1 being that a differential value generated from the event estimated to be the R-wave is a negative constant C₃ value or less;

generating a mark display signal for providing a mark to an event estimated to be an R-wave on a display unit when or after the condition 1 is satisfied; and

generating an enable signal when or after the step of generating a mark display signal.

[15] The method for generating a defibrillation signal according to above [14], including the steps of:

determining whether condition 2 is satisfied, the condition 2 being that a peak value of a differentiated waveform (hereinafter, simply referred to as “positive wave”) is a positive constant C₁ value or more, the differentiated waveform being a set of differential values generated from a portion of the event estimated to be the R-wave that corresponds to a rise phase occurring before the peak of the event estimated to be the R-wave; and

generating a mark display signal for providing a mark to an event estimated to be an R-wave on the display unit when or after the condition 2 is satisfied and the condition 1 is satisfied.

[16] The method for generating a defibrillation signal according to above [15], including the steps of:

determining whether condition 3 is satisfied, the condition 3 being that a period of time during which the differential values of the positive wave are a positive constant C₂ value or more is measured and is 10 milliseconds or more and 80 milliseconds or less, the positive constant C₂ value being smaller than the C₁ value; and

generating a mark display signal for providing a mark to an event estimated to be an R-wave on the display unit when or after the condition 2 and the condition 3 are satisfied and the condition 1 is satisfied.

[17] The method for generating a defibrillation signal according to any one of above [14] to [16], including the steps of:

determining whether condition 4 is satisfied, the condition 4 being that a period of time from when a differential value generated from an event estimated to be an R-wave (hereinafter, simply referred to as “Rₙ₋₁-wave”) immediately before the event estimated to be the R-wave (hereinafter, simply referred to as “Rₙ-wave”) reaches the C₃ value until the differential value generated from the Rₙ-wave reaches the C₃ value is measured and is 50 milliseconds or more; and

generating a mark display signal for providing a mark to an event estimated to be an R-wave on the display unit when or after the condition 4 is satisfied and the condition 1 is satisfied.

### Effects of the Invention

According to the present invention, by the above-described configuration, a new electric device for defibrillation and a method for generating a defibrillation signal can be provided.

### BRIEF DESCRIPTION OF THE DRAWINGS

FIG. 1 is a schematic diagram showing a configuration of a defibrillation catheter system including an electric device for defibrillation according to a first embodiment of the present invention.

FIG. 2 is a diagram showing examples of an electrocardiogram waveform displayed on a display unit of an electrocardiograph, and a differentiated waveform which is a set of differential values of the electrocardiogram waveform.

FIG. 3 is a diagram showing another example of a differentiated waveform which is a set of differential values of an electrocardiogram waveform.

FIG. 4 is a block diagram of the defibrillation catheter system including the electric device for defibrillation according to the first embodiment of the present invention.

FIG. 5 is a block diagram of an electric device for defibrillation according to a second embodiment of the present invention.

FIG. 6 is a flowchart showing an example of a processing procedure performed by the electric device for defibrillation according to the second embodiment of the present invention.

### MODE FOR CARRYING OUT THE INVENTION

Although the present invention will be more specifically described below based on the following embodiments, needless to say, the present invention is not limited to the following embodiments and it is, of course, possible to implement the present invention by appropriately adding changes thereto that come within the meaning described above or later, and all those changes are embraced in the technical scope of the present invention. Note that although in each drawing, reference signs for members, etc., may be omitted for the sake of convenience, in that case, the specification or other drawings are to be referred to. Note also that the sizes of various members, etc., in the drawings may differ from their actual sizes because helping understanding of the features of the present invention is prioritized.

An electric device for defibrillation of the present invention includes an electrocardiogram waveform input unit and an enable signal generating unit, and is characterized in that the electric device for defibrillation is controlled to generate an enable signal from the enable signal generating unit after a peak of an event estimated to be an R-wave of an electrocardiogram waveform which is obtained from a human body and inputted from the electrocardiogram waveform input unit is surpassed and when or after the following condition 1 is satisfied:

(Condition 1) A differential value generated from the event estimated to be an R-wave is a negative constant C₃ value or less.

As described above, in the electric device for defibrillation, a threshold value (negative constant C₃ value) is provided for a differential value of a portion of an event estimated to be an R-wave of an electrocardiogram waveform that corresponds to a fall phase occurring after a peak of the event estimated to be an R-wave is surpassed, and an electric device for defibrillation having this configuration does not exist conventionally. Furthermore, by having this configuration, a determination as to whether an application-target waveform is an R-wave is facilitated, and application of a voltage associated with erroneous detection of an R-wave can be easily avoided.

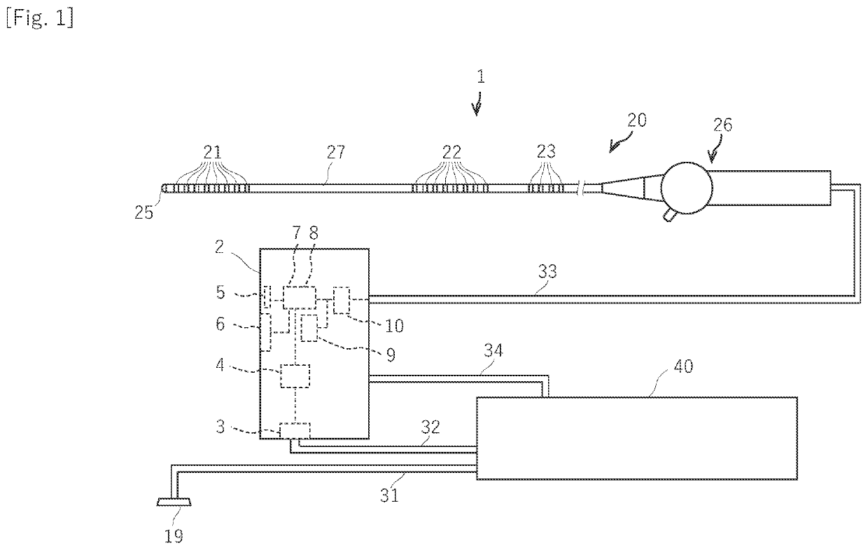

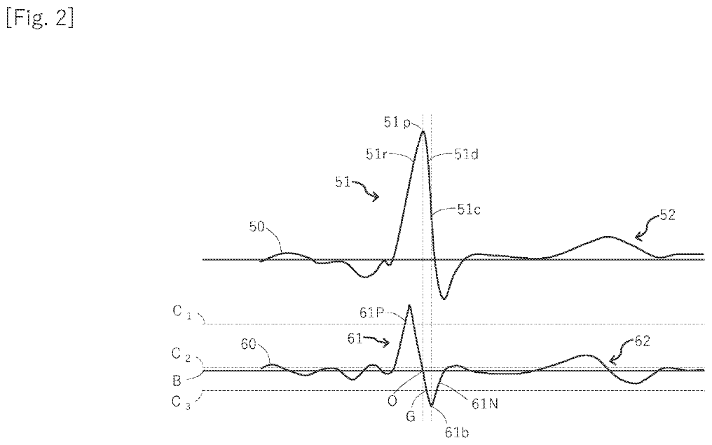

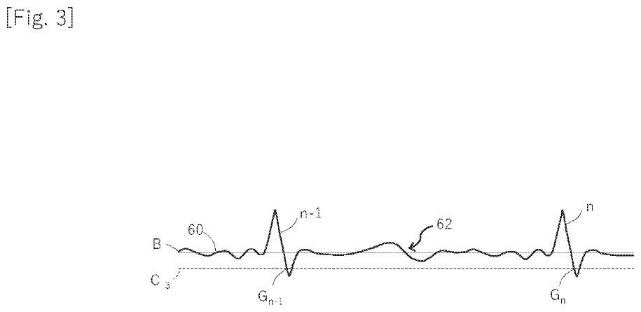

With reference to FIGS. 1 to 3, configurations of an electric device for defibrillation and a defibrillation catheter system including the electric device for defibrillation according to a first embodiment of the present invention will be described below. FIG. 1 is a schematic diagram showing a configuration of a defibrillation catheter system including an electric device for defibrillation according to the first embodiment of the present invention. FIG. 2 is a diagram showing examples of an electrocardiogram waveform displayed on a display unit (not shown) of an electrocardiograph, and a differentiated waveform which is a set of differential values of the electrocardiogram waveform. A horizontal axis of the electrocardiogram waveform of FIG. 2 represents time (seconds) and a vertical axis represents a voltage difference (mV). A dashed line C₁ extending in a time-axis direction of the differentiated waveform of FIG. 2 is a line whose value (differential value) on the vertical axis is a positive constant C₁, a dashed line C₂ extending in the time-axis direction is a line whose value (differential value) on the vertical axis is a positive constant C₂ value, and a dashed line C₃ extending in the time-axis direction is a line whose value (differential value) on the vertical axis is a negative constant C₃ value. In FIG. 2, a solid line B extending in the time-axis direction is a baseline of the differentiated waveform. FIG. 3 is a diagram showing another example of a differentiated waveform which is a set of differential values of an electrocardiogram waveform.

An electric device for defibrillation 2 of FIG. 1 includes an electrocardiogram waveform input unit 3 and an enable signal generating unit 7. To the electric device for defibrillation 2 is inputted, for example, an electrocardiogram waveform which is obtained from a body-surface electrode 19 disposed on a human's body surface, through the electrocardiogram waveform input unit 3 via an electrocardiograph 40, etc. Furthermore, the electric device for defibrillation 2 is controlled to generate an enable signal from the enable signal generating unit 7 after a peak 51p of an event 51 which is estimated to be an R-wave of an electrocardiogram waveform 50 such as that shown in FIG. 2 is surpassed and when or after the following condition 1 is satisfied:

(Condition 1) A differential value generated from the event 51 estimated to be an R-wave is the negative constant C₃ value or less.

A differentiated waveform 60 of FIG. 2 is an example of a set of differential values generated from the electrocardiogram waveform 50, and a negative wave 61N of the differentiated waveform 60 corresponds to a set of differential values generated from a portion of the event 51 estimated to be an R-wave of the electrocardiogram waveform 50 that corresponds to a fall phase 51d occurring after the peak 51p of the event 51 estimated to be an R-wave. The timing of generating an enable signal will be described below with reference to the differentiated waveform 60 of FIG. 2. A point G of the differentiated waveform 60 corresponds to a point in time when a differential value reaches the negative constant C₃ value, and the electric device for defibrillation 2 is controlled to generate an enable signal at the point G or at any timing after the point G. In FIG. 2, a waveform that falls below the point G is only present at the negative wave 61N which is generated from the event 51 estimated to be an R-wave, and thus, a determination as to whether an application-target waveform is an R-wave can be easily made using condition 1. By setting such a threshold value (negative constant C₃ value), application of a voltage associated with erroneous detection of an R-wave can be easily avoided. Meanwhile, it is preferable that the electric device for defibrillation 2 be controlled to generate an enable signal before a peak 61b of the negative wave 61N. By this, defibrillation can be easily completed during an absolute refractory period. In addition, it is preferable that the electric device for defibrillation 2 be controlled to generate an enable signal within 60 milliseconds from when a differential value reaches the negative constant C₃ value (point G), and it is more preferable that the electric device for defibrillation 2 be controlled to generate an enable signal within 50 milliseconds, and it is further preferable that the electric device for defibrillation 2 be controlled to generate an enable signal within 10 milliseconds, and it is most preferable that the electric device for defibrillation 2 be controlled to generate an enable signal when a differential value reaches the negative constant C₃ value. Note that the peak 61b of the negative wave 61N corresponds to an inflection point 51c of the fall phase 51d of the event 51 estimated to be an R-wave.

The enable signal is not particularly limited as long as the enable signal is a signal for application of a voltage for defibrillation. Examples of the enable signal include an enable signal for charging of a power supply unit 9 which will be described later, an enable signal for generation of a pulse voltage, an enable signal for voltage application, and an enable signal for switching-on of a switching unit 10 which will be described later. When the above-described condition 1 is satisfied, the enable signal generating unit 7 generates at least one of those enable signals. On the other hand, regardless of the above-described condition 1, for example, by operating an operating unit 6 which will be described later, some of those enable signals may be generated. Note that the enable signal generating unit 7 is not limited to being provided in an arithmetic processing control unit 8 which will be described later, and may be provided in the power supply unit 9, etc.

Examples of the above-described differential value generated from the event 51 estimated to be an R-wave include a differential value obtained through a differentiating circuit 4 which will be described later and a differential value obtained by general differential computation. In addition, it is preferable that the differential value generated from the event 51 estimated to be an R-wave be a first-order differential value. The first-order differential value requires a shorter time to be generated than a second-order differential value, and thus, a period of time from obtaining of electrocardiogram information to generation of an enable signal can be reduced. The differentiated waveform 60 may be generated from a normalized electrocardiogram waveform which has been generated by normalization of the electrocardiogram waveform 50. In the normalization, a voltage difference at no signal may be converted into 0V, and a voltage difference at a peak of the event 51 estimated to be an R-wave may be converted into 1V, so as to generate the normalized electrocardiogram waveform. The normalization may be carried out by the electrocardiograph 40, the arithmetic processing control unit 8, a first arithmetic processing control unit 72 mentioned later, a second arithmetic processing control unit 75 mentioned later, etc. In this case, the unit of the vertical axis of the normalized electrocardiogram waveform may be [V], the unit of the horizontal axis of the normalized electrocardiogram waveform may be [s], the unit of the vertical axis of the differential wave may be [V/s], and the unit of the horizontal axis of the differential wave may be [s].

The above-described negative constant C₃ value is, for example, a value that falls below the value (differential value) on the vertical axis of the baseline B in the differentiated waveform 60 of FIG. 2. Note that the value (differential value) on the vertical axis of the baseline B is the same as the value (differential value) on the vertical axis of a point O of the differentiated waveform 60 which is a portion corresponding to the peak 51p of the event 51 estimated to be an R-wave. In addition, the negative constant C₃ value may be a value that varies depending on the type of the differentiating circuit 4, etc. The preferable range of the negative constant C₃ value is as follows. When the differentiated waveform 60 is generated from a normalized electrocardiogram waveform which has been generated by normalizing the electrocardiogram waveform 50, the normalizing including converting a voltage difference at no signal into 0V and a voltage difference at a peak of the event estimated to be an R-wave into 1V, the negative constant C₃ value is preferably −1 to −100 V/s and more preferably −5 to −80 V/s.

It is preferable that the electrocardiogram waveform 50 be a waveform obtained using lead II that facilitates detection of an event estimated to be an R-wave. Note, however, that instead of lead II, the electrocardiogram waveform 50 may be obtained using other leads depending on the orientation of a patient's heart. For example, when an electrocardiogram waveform is obtained using 12 leads, the electrocardiogram waveform 50 may be a waveform obtained using lead V1, lead V2, lead V3, lead V4, lead V5, lead V6, lead I, lead II, lead III, lead aVR, lead aVL, or lead aVF. In addition, the electrocardiogram waveform 50 may be a waveform using the average of two or more leads, a waveform using the average of three or more leads, or a waveform using the average of 12 leads.

It is preferable that the electric device for defibrillation 2 be controlled to generate an enable signal when or after the following condition 2 is satisfied and condition 1 is satisfied:

(Condition 2) A peak value of a differentiated waveform (hereinafter, simply referred to as “positive wave 61P”) which is a set of differential values generated from a portion of the event 51 estimated to be an R-wave that corresponds to a rise phase 51r occurring before the peak 51p of the event 51 estimated to be an R-wave, is the positive constant C₁ value or more.

In the electric device for defibrillation 2, a threshold value (positive constant C₁ value) is provided for a peak value of the positive wave 61P as shown in the above-described condition 2, which makes it easier to determine whether an application-target waveform is an R-wave, and thus, application of a voltage associated with erroneous detection of an R-wave can be easily avoided.

The above-described positive constant C₁ value is, for example, a value that exceeds the value (differential value) on the vertical axis of the baseline B in the differentiated waveform 60 of FIG. 2. In addition, the positive constant C₁ value may be a value that varies depending on the type of the differentiating circuit 4, etc. The preferable range of the positive constant C₁ value is as follows. When the differentiated waveform 60 is generated from a normalized electrocardiogram waveform which has been generated by normalizing the electrocardiogram waveform 50, the normalizing including converting a voltage difference at no signal into 0V and a voltage difference at a peak of the event estimated to be an R-wave into 1V, the positive constant C₁ value is preferably 1 to 100 V/s and more preferably 5 to 80 V/s.

It is preferable that the electric device for defibrillation 2 be controlled to generate an enable signal when or after the above-described condition 2 and the following condition 3 are satisfied and the above-described condition 1 is satisfied:

(Condition 3) A period of time during which the differential values of the positive wave 61P are the positive constant C₂ value or more is measured and is 10 milliseconds or more and 80 milliseconds or less, the positive constant C₂ value being smaller than the C₁ value.

In the electric device for defibrillation 2, as shown in the above-described condition 3, there is provided a threshold value for an upper limit to a period of time during which the differential values of the positive wave 61P are the positive constant C₂ value or more, by which erroneous detection of an R-wave can be easily avoided. Specifically, a differentiated waveform 62 generated from a T-wave 52 of a patient may be similar to a differentiated waveform 61 generated from the event 51 estimated to be an R-wave, but by the above-described threshold value, the differentiated waveform 62 which is derived from the T-wave 52 whose period of time defined in the above-described condition 3 is long can be easily excluded from a voltage application target. The period of time is more preferably 70 milliseconds or less and further preferably 60 milliseconds or less. On the other hand, by the period of time being 10 milliseconds or more, high-frequency noise with a short peak width can be easily excluded. As a result, R-wave detection sensitivity can be improved. The period of time is more preferably 15 milliseconds or more and further preferably 20 milliseconds or more.

The above-described positive constant C₂ value is, for example, a value that exceeds the value (differential value) on the vertical axis of the baseline B in the differentiated waveform 60 of FIG. 2. In addition, the positive constant C₂ value may be a value that varies depending on the type of the differentiating circuit 4, etc. The preferable range of the positive constant C₂ value is as follows. When the differentiated waveform 60 is generated from a normalized electrocardiogram waveform which has been generated by normalizing the electrocardiogram waveform 50, the normalizing including converting a voltage difference at no signal into 0V and a voltage difference at a peak of the event estimated to be an R-wave into 1V, the positive constant C₂ value is preferably 0.4 to 40 V/s and more preferably 1 to 20 V/s. Note that the C₂ value is smaller than the C₁ value.

The electric device for defibrillation 2 may be controlled to generate an enable signal when condition 2 is not satisfied and when or after condition 3 and condition 1 are satisfied.

It is preferable that the electric device for defibrillation 2 be controlled to generate an enable signal when or after the following condition 4 is satisfied and condition 1 is satisfied:

(Condition 4) A period of time from when a differential value generated from an event estimated to be an R-wave (hereinafter, simply referred to as “Rₙ₋₁-wave”) immediately before an event estimated to be an R-wave (hereinafter, simply referred to as “Rₙ-wave”) reaches the negative constant C₃ value until a differential value generated from the Rₙ-wave reaches the negative constant C₃ value is measured and is 50 milliseconds or more.

With reference to FIG. 3, condition 4 will be described below. FIG. 3 is a diagram showing another example of a differentiated waveform which is a set of differential values of an electrocardiogram waveform. A differentiated waveform 60 of FIG. 3 includes a differentiated waveform n which is a set of differential values generated from an Rₙ-wave (not shown) of an electrocardiogram waveform; and a differentiated waveform n−1 which is a set of differential values generated from an Rₙ₋₁-wave (not shown) immediately before the Rₙ-wave. Satisfying condition 4 indicates that in a case of FIG. 3, a period of time from a point Gₙ₋₁ at which a value (differential value) on the vertical axis of the differentiated waveform n−1 reaches the negative constant C₃ value to a point Gₙ at which a value (differential value) on the vertical axis of the differentiated waveform n reaches the negative constant C₃ value (which may be hereinafter referred to as the period of time Gₙ₋₁-Gₙ) is 50 milliseconds or more. By setting the period of time to 50 milliseconds or more, a differentiated waveform 62 which is derived from a T-wave can be easily excluded from a voltage application target, and thus, application of a voltage associated with erroneous detection can be easily avoided. The period of time Gₙ₋₁-Gₙ is more preferably 100 milliseconds or more, further preferably 200 milliseconds or more, even more preferably 240 milliseconds or more, and particularly preferably 260 milliseconds or more. On the other hand, an upper limit to the period of time Gₙ₋₁-Gₙ is not particularly limited, but for example, the period of time Gₙ₋₁-Gₙ may be 2 seconds or less, 1 second or less, 800 milliseconds or less, 600 milliseconds or less, 400 milliseconds or less, or 350 milliseconds or less.

The electric device for defibrillation 2 may be controlled to generate an enable signal when or after at least either one of condition 2 and condition 3, condition 4, and condition 1 are satisfied.

It is preferable that the electric device for defibrillation 2 be controlled to generate an enable signal when or after the following condition 5 is satisfied and condition 1 is satisfied:

(Condition 5) A period of time from the point O of the differentiated waveform 60 which is a portion corresponding to the peak 51p of the event 51 estimated to be an R-wave to the point G at which a differential value reaches the negative constant C₃ value is measured and is 2 (milliseconds) or more and 20 (milliseconds) or less.

In the electric device for defibrillation 2, a threshold value is provided for the period of time from the point O to point G of the differentiated waveform 60 as shown in the above-described condition 5, by which erroneous detection of an R-wave can be easily avoided.

The electric device for defibrillation 2 may be controlled to generate an enable signal when or after at least one condition selected from a group consisting of condition 2, condition 3, and condition 4, condition 5, and condition 1 are satisfied.

It is preferable that the negative constant C₃ value, the positive constant C₂ value, the positive constant C₁ value, the threshold value for the period of time Gₙ₋₁-Gₙ, and the threshold value for the period of time from the point O to the point G each be stored in a memory which will be described later or be set in a comparator. In addition, these values do not need to be stored in the same memory and may be stored in different memories. In addition, these values do not need to be stored in the same comparator and may be stored in different comparators.

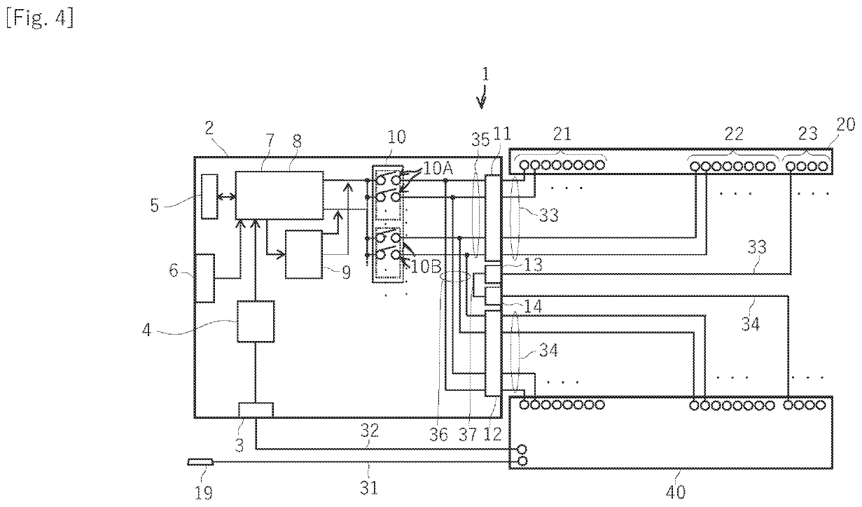

A configuration for generation of an enable signal of the electric device for defibrillation 2 is mainly described above. With reference to FIGS. 1 and 2, configurations of the electric device for defibrillation 2 and the defibrillation catheter system 1 including the electric device for defibrillation 2 according to the first embodiment will be specifically described below. FIG. 4 is a block diagram of the defibrillation catheter system 1 including the electric device for defibrillation 2 according to the first embodiment.

In the defibrillation catheter system 1 of FIGS. 1 and 4, electrocardiogram information obtained from the body-surface electrode 19 disposed on the human's body surface is transmitted to the electrocardiograph 40 through a first conducting wire 31. An electrode that obtains electrocardiogram information is not limited to a body-surface electrode and may be an electrode for measuring an intracardiac potential, but it is preferable to use a body-surface electrode because of its excellent R-wave detection sensitivity. For the body-surface electrode, it is preferable to use electrodes for 12 leads.

The electric device for defibrillation 2 of FIGS. 1 and 4 includes a first connecting unit 11 connected to a plurality of electrodes which are provided on a distal side of a catheter 20; a second connecting unit 12 connected to the electrocardiograph 40; the power supply unit 9 that generates a voltage to be applied; and the switching unit 10 that is connected to the power supply unit 9 and switches to an application mode in which a voltage is applied. In addition, the first connecting unit 11 is connected to the power supply unit 9 through the switching unit 10 and connected to the second connecting unit 12 without the switching unit 10 therebetween. By the first connecting unit 11 being connected to the second connecting unit 12 without the switching unit 10 therebetween, even upon defibrillation, a local potential of each electrode can be measured.

In addition, the electric device for defibrillation 2 includes the electrocardiogram waveform input unit 3, and information on an electrocardiogram waveform outputted from the electrocardiograph 40 is inputted into the electric device for defibrillation 2 through the electrocardiogram waveform input unit 3 via a second conducting wire 32, etc. The electrocardiogram waveform input unit 3 is not particularly limited, but is preferably one that can withstand a discharge of 5 kV which is inputted through a 50Ω resistor. The electrocardiogram waveform input unit 3 may include a connector configured to be connected to the electrocardiograph 40 via at least one conducting wire, a connector configured to be connected to the body-surface electrode 19 via at least one conducting wire, etc.

The electrocardiogram waveform inputted through the electrocardiogram waveform input unit 3 is transmitted to the arithmetic processing control unit 8 through the differentiating circuit 4. The arithmetic processing control unit 8 determines whether a transmitted differentiated waveform 60 satisfies conditions related to threshold values such as the negative constant C₃ value stored in a memory 5, i.e., condition 1, etc., and when condition 1, etc., are satisfied, the enable signal generating unit 7 in the arithmetic processing control unit 8 can generate an enable signal for voltage application. The enable signal is transmitted to the power supply unit 9, and direct-current voltages having different, positive and negative, polarities can be applied to a first electrode group 21 and a second electrode group 22. An energization waveform may be a biphasic waveform in which the polarity is reversed midway or may be a monophasic waveform with constant polarity, but the biphasic waveform is preferable because the biphasic waveform is said to be able to deliver a stimulus with a smaller energy. Energization energy to be provided to a living body can be set to, for example, 1 J or more and 30 J or less.

For the differentiating circuit 4 and the memory 5, publicly known ones can be used, and the differentiating circuit 4 and the memory 5 may be provided in the arithmetic processing control unit 8 or may be provided separately. In addition, the differentiating circuit 4 and the memory 5 may be, for example, integrated into one unit in an FPGA which will be described later. Note that the electric device for defibrillation 2 may include, though not shown, a display unit that displays an electrocardiogram waveform, and a mark may be displayed for an event estimated to be an R-wave on the display unit. For the display unit and the mark, description of a display unit 73 of a second embodiment can be referred to.

It is preferable that the power supply unit 9 include, for example, a power supply, a booster circuit that boosts a direct-current voltage, a charging circuit, a capacitor that charges a voltage to be applied, and a waveform generation circuit that generates a pulse voltage. Note that at least one of these components may be provided outside the power supply unit 9. The location of the power supply unit 9 is not particularly limited. For example, the power supply unit 9 may be provided outside the arithmetic processing control unit 8 as shown in FIG. 4 or may be provided in the arithmetic processing control unit 8.

When the electrocardiogram waveform inputted through the electrocardiogram waveform input unit 3 satisfies condition 1, etc., the enable signal generating unit 7 in the arithmetic processing control unit 8 may be controlled to generate an enable signal for switching-on. The enable signal is transmitted to first switches 10A and second switches 10B in the switching unit 10, by which the first switches 10A and the second switches 10B can be changed from off state to on state, and as a result, the first electrode group 21 and the second electrode group 22 can be energized. Note that when the switches included in the switching unit 10 are in off state as shown in FIG. 4, the first electrode group 21 and the second electrode group 22 are insulated from the power supply unit 9, and thus, without performing defibrillation, an intracardiac potential can be measured using the first electrode group 21 and the second electrode group 22.

At least one of functions of the electric device for defibrillation 2, e.g., the functions of the electrocardiogram waveform input unit 3, the differentiating circuit 4, the memory 5, the enable signal generating unit 7, the arithmetic processing control unit 8, the power supply unit 9, and/or the switching unit 10, may be implemented by hardware or may be implemented by software. Examples of the hardware include a logic circuit formed in an integrated circuit such as a large scale integration (LSI), an application specific integrated circuit (ASIC), or a field-programmable gate array (FPGA).

The electric device for defibrillation 2 may include a computer that performs instructions of a program which is software for implementing at least one of the functions of the electrocardiogram waveform input unit 3, the differentiating circuit 4, the memory 5, the enable signal generating unit 7, the arithmetic processing control unit 8, the power supply unit 9, and/or the switching unit 10. It is preferable that the computer include a processor and a computer-readable recording medium having stored therein the above-described program. By the processor executing the program stored in the computer-readable recording medium, the above-described functions are implemented. For the processor, a central processing unit (CPU) can be used. For the recording medium, a read only memory (ROM), etc., can be used. In addition, the recording medium can also include a random access memory (RAM). The above-described program may be supplied to the above-described computer through any transmission medium through which the program can be transmitted. Examples of the transmission medium include a communication network and a communication line.

In addition, it is preferable that the electric device for defibrillation 2 of FIGS. 1 and 3 be provided with the operating unit 6 for performing various operations such as the activation and stopping of the electric device for defibrillation 2, setting of the amount of energy to be applied, charging, application of a voltage, and selection of application electrodes. For the operating unit 6, publicly known input means such as button switches and levers can be used. It is preferable that the operating unit 6 be connected to the arithmetic processing control unit 8, by which an input signal from the operating unit 6 is transmitted to the arithmetic processing control unit 8. Note that some of the above-described enable signals may be generated by operating the operating unit 6.

It is preferable that the first electrode group 21 and the second electrode group 22 be connected to the electrocardiograph 40 without the switching unit 10 therebetween, and it is more preferable that the first electrode group 21 and the second electrode group 22 be connected to the electrocardiograph 40 without any switch unit therebetween. By this, the first electrode group 21 and the second electrode group 22 can always be connected to the electrocardiograph 40, and thus, various types of treatment can be easily given while checking on an intracardiac potential displayed on the display unit (not shown) of the electrocardiograph 40.

The switching unit 10 may include one or two or more switches. It is preferable that as shown in FIG. 4, the switching unit 10 include the plurality of first switches 10A connected in parallel to each other and the plurality of second switches 10B connected in parallel to each other. When the catheter 20 includes the first electrode group 21 and the second electrode group 22, it is preferable that each electrode in the first electrode group 21 be connected to the power supply unit 9 through a corresponding first switch 10A, and each electrode in the second electrode group 22 be connected to the power supply unit 9 through a corresponding second switch 10B. Namely, it is preferable that the first electrode group 21 and the second electrode group 22 be connected to the power supply unit 9 through different switches. By this, the electrode groups can be electrically separated from each other, and thus, the electrode groups can obtain an intracardiac potential independently of each other.

As shown in FIG. 1, the electric device for defibrillation 2 may include a third electrode group 23 which is electrodes dedicated to measurement of an intracardiac potential, more on a proximal side than the first electrode group 21 and the second electrode group 22. Since the third electrode group 23 is located on the proximal side, the third electrode group 23 can be disposed, for example, at a location corresponding to the ascending aorta. It is preferable that the third electrode group 23 not be connected to the power supply unit 9. By this, the third electrode group 23 can be easily used as electrodes dedicated to measurement of an intracardiac potential.

The number of electrodes included in each electrode group is not particularly limited, and the electrode groups may have the same number of electrodes. It is preferable that particularly, the number of electrodes included in the first electrode group 21 be the same as the number of electrodes included in the second electrode group 22. By this, the surface areas of the first electrode group 21 and the second electrode group 22 can be easily made identical. By the electrodes in the first electrode group 21 and the electrodes in the second electrode group 22 having the same surface area and identical numbers of electrodes being disposed at equal intervals, efficient defibrillation can be performed and the accuracy of measurement of an intracardiac electrocardiogram can be improved.

It is preferable that the number of electrodes included in the third electrode group 23 be less than or equal to each of the number of electrodes included in the first electrode group 21 and the number of electrodes included in the second electrode group 22. For example, the number of electrodes in each of the first electrode group 21 and the second electrode group 22 can be eight, and the number of electrodes in the third electrode group 23 can be four. By thus setting the number of electrodes in the third electrode group 23, a potential at a location corresponding to the ascending aorta can be suitably measured.

It is preferable that each electrode group be present in a region that is half or more of the outer periphery of a resin tube 27, and it is more preferable that each electrode group be formed in a ring shape. By thus forming electrodes, the contact area with the heart increases, making it easier to measure an intracardiac potential or deliver an electrical stimulus.

Each electrode group includes any conductive material such as platinum or stainless steel, but in order to facilitate grasping of the locations of electrodes under X-ray radioscopy, it is preferable that each electrode group include a radiopaque material such as platinum.

As shown in FIG. 1, an end tip 25 may be provided at a distal end portion of the catheter 20. It is preferable that the end tip 25 have a tapered portion whose outside diameter decreases toward the distal side. The end tip 25 may be made of a conductive material. By this, the end tip 25 can function as an electrode. In addition, the end tip 25 may be made of a polymeric material, and in order to protect body tissues from contact with the catheter 20, the hardness of the end tip 25 may be lower than the hardness of the resin tube 27.

In the lumen of the resin tube 27 there may be disposed an operation wire or a spring member for bending the distal side of the catheter 20. Specifically, it is preferable that a distal end portion of the operation wire be fixed at a distal end portion of the resin tube 27 or at the end tip 25, and a proximal end portion of the operation wire be fixed at a handle 26 which will be described later.

As shown in FIG. 4, it is preferable that third conducting wires 33 (lead wires) be connected to each electrode group. Other end portions of the third conducting wires 33 connected to the first electrode group 21 and the second electrode group 22 are preferably connected to the first connecting unit 11 of the electric device for defibrillation 2. Other end portions of the third conducting wires 33 connected to the third electrode group 23 are preferably connected to a third connecting unit 13 of the electric device for defibrillation 2. The third conducting wires 33 each may be a plurality of conducting wires connected together by a connecting member such as a connector.

It is preferable that the third connecting unit 13 be connected to a fourth connecting unit 14 through a seventh conducting wire 37. Here, the seventh conducting wire 37 may be a wiring material or may be a part of a wiring pattern provided on a printed circuit board.

It is preferable that the first connecting unit 11 be connected to the switching unit 10 through fifth conducting wires 35. By this, the first electrode group 21 and the second electrode group 22 are connected to the power supply unit 9, and thus, application of a voltage can be performed. The first electrode group 21 and the second electrode group 22 may be connected to the power supply unit 9 through different connecting members such as connectors.

It is preferable that other ends of fourth conducting wires 34 which are connected to input terminals of the electrocardiograph 40 corresponding to the first electrode group 21 and the second electrode group 22 be connected to the second connecting unit 12. In addition, it is preferable that the second connecting unit 12 be connected to the fifth conducting wires 35 by sixth conducting wires 36. It is preferable that the fifth conducting wires 35 and the sixth conducting wires 36 not be provided with a switch unit. By this, even upon defibrillation, an intracardiac potential can be measured through the first electrode group 21 and the second electrode group 22. Here, the fifth conducting wires 35 and the sixth conducting wires 36 may be wiring materials or may be a part of a wiring pattern provided on a printed circuit board.

As shown in FIG. 1, the handle 26 that is grasped by a user upon operation of the catheter 20 may be provided on a proximal side of the resin tube 27. The shape of the handle 26 is not particularly limited, but in order to reduce stress concentration at a location where the resin tube 27 is connected to the handle 26, it is preferable that the handle 26 be formed in a cone shape whose outside diameter decreases toward the distal side.

The electrocardiograph 40 measures an intracardiac potential through various electrodes. For the electrocardiograph 40, a publicly known one can be used.

Though not shown, the electric device for defibrillation 2 may include an electrode selection switch that selects an electrode to which a voltage is applied. By this, an electrical stimulus can be delivered only to a specific electrode. A location where the electrode selection switch is provided is not particularly limited, but it is preferable that the electrode selection switch be connected to the power supply unit 9, and it is more preferable that the electrode selection switch be provided in the arithmetic processing control unit 8. The electrode selection switch may be provided separately from switches (e.g., the first switches 10A and the second switches 10B) included in the switching unit 10, or at least one of the switches included in the switching unit 10 may be an electrode selection switch. In addition, though not shown, the electric device for defibrillation 2 may be provided with a switch for safety. By this, upon failure of the switching unit 10, etc., a fail-safe function that can suppress unintentional application of a voltage to a patient can be provided. It is preferable that the safety switch be connected between the switching unit 10 and the power supply unit 9, and it is more preferable that the safety switch be connected between the arithmetic processing control unit 8 and the switching unit 10. In addition, though not shown, the electric device for defibrillation 2 may be provided with a protection circuit that absorbs high voltage occurring upon a shutdown of switches. By this, breakage of each switch can be prevented. In addition, though not shown, in the electric device for defibrillation 2, an overvoltage protection circuit that protects the electrocardiograph 40 from overvoltage may be provided between the power supply unit 9 and the electrocardiograph 40. By this, the electrocardiograph 40 can be prevented from breaking down by application of overvoltage. In addition, though not shown, the electric device for defibrillation 2 may include an impedance measurement circuit. It is preferable that the impedance measurement circuit be connected, for example, between the first electrode group 21 and the second electrode group 22 to measure impedance between the first electrode group 21 and the second electrode group 22.

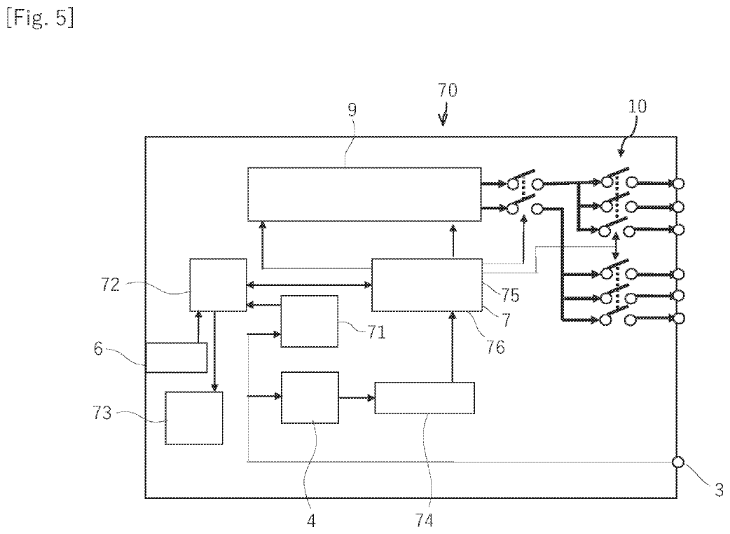

Next, with reference to FIG. 5, a configuration of an electric device for defibrillation 70 according to a second embodiment will be described in detail. FIG. 5 is a block diagram of the electric device for defibrillation 70 according to the second embodiment. Note that the same components as those of the electric device for defibrillation 2 according to the first embodiment are given the same reference signs and description thereof is omitted.

It is preferable that in the electric device for defibrillation 70 according to the second embodiment, as shown in FIG. 5, electrocardiogram information inputted through the electrocardiogram waveform input unit 3 pass through an A/D converter 71 and a first arithmetic processing control unit 72 (CPU), by which an electrocardiogram waveform is displayed on the display unit 73. On the other hand, the electrocardiogram information inputted through the electrocardiogram waveform input unit 3 passes through the differentiating circuit 4, generating a differentiated waveform. It is preferable that the differentiated waveform be then transmitted to a comparator 74 in which the negative constant C₃ value, etc., are set, and if condition 1, etc., are satisfied, then a signal be transmitted to a second arithmetic processing control unit 75 (FPGA), and the second arithmetic processing control unit 75 (FPGA) generate a mark display signal, and after transmitting the mark display signal to the first arithmetic processing control unit 72 (CPU), a mark be displayed for an event estimated to be an R-wave on the display unit 73. Examples of the shape of the mark include polygons such as circle, triangle, and rectangle, and linear. Examples of a location where the mark is displayed include a peak of an event estimated to be an R-wave. In addition, the mark display signal may be any signal as long as a mark is displayed for an event estimated to be an R-wave on the display unit 73, and the first arithmetic processing control unit 72 (CPU) may generate the mark display signal.

As described above, it is preferable that the electric device for defibrillation 70 include the display unit 73 that displays an electrocardiogram waveform, and be controlled to generate a mark display signal for providing a mark to an event estimated to be an R-wave on the display unit 73 from a mark display signal generating unit 76 after a peak of the event estimated to be an R-wave is surpassed and when or after the following condition 1 is satisfied. By thus providing a mark to an event estimated to be an R-wave on the display unit 73, an operator can visually check the state of the R-wave.

(Condition 1) A differential value generated from the event estimated to be an R-wave is the negative constant C₃ value or less.

Furthermore, it is preferable that the electric device for defibrillation 70 be controlled to generate a mark display signal when or after the following condition 2 is satisfied and condition 1 is satisfied:

(Condition 2) A peak value of a differentiated waveform (hereinafter, simply referred to as “positive wave 61P”) which is a set of differential values generated from a portion of the event estimated to be an R-wave that corresponds to a rise phase occurring before a peak of the event estimated to be an R-wave, is the positive constant C₁ value or more.

Furthermore, it is preferable that the electric device for defibrillation 70 be controlled to generate a mark display signal when or after condition 2 and the following condition 3 are satisfied and condition 1 is satisfied:

(Condition 3) A period of time during which the differential values of the positive wave 61P are the C₂ value or more is measured and is 10 milliseconds or more and 80 milliseconds or less, the C₂ value being smaller than the C₁ value.

Furthermore, it is preferable that the electric device for defibrillation 70 be controlled to generate a mark display signal when or after the following condition 4 is satisfied and condition 1 is satisfied:

(Condition 4) A period of time from when a differential value generated from an event estimated to be an R-wave (hereinafter, simply referred to as “Rₙ₋₁-wave”) immediately before an event estimated to be an R-wave (hereinafter, simply referred to as “Rₙ-wave”) reaches the C₃ value until a differential value generated from the Rₙ-wave reaches the C₃ value is measured and is 50 milliseconds or more.

Note that for details of these conditions 1 to 4, description of the electric device for defibrillation 2 according to the first embodiment can be referred to.

In addition, it is preferable that in the electric device for defibrillation 70, by operating the operating unit 6, switching from a no-permission mode to a permission mode be able to be performed in the second arithmetic processing control unit 75 (FPGA). In addition, at the same time as switching from the no-permission mode to the permission mode, the amount of energy to be applied may be able to be set, or charging of energy to be applied into the capacitor may start, or the charging may be completed. Furthermore, after completing the charging, a pulse voltage may be automatically generated. The no-permission mode is a mode in which even if the above-described condition 1, etc., are satisfied, an enable signal for defibrillation is not generated, and the permission mode is a mode in which when the above-described condition 1, etc., are satisfied, an enable signal for defibrillation is generated. By this, the operator can set the no-permission mode when the state of a patient is bad, and can switch to the permission mode after the state of the patient gets better, and thus, defibrillation can be easily performed. The enable signal for defibrillation is not particularly limited as long as the enable signal is a signal for application of a voltage for defibrillation, and examples of the enable signal include an enable signal for charging of the power supply unit 9, an enable signal for generation of a pulse voltage, an enable signal for voltage application, and an enable signal for switching-on of the switching unit 10. For other details on the enable signal for defibrillation, description of the first embodiment can be referred to.

In addition, it is preferable that the electric device for defibrillation 70 be configured such that electrocardiogram information inputted through the electrocardiogram waveform input unit 3 passes through the differentiating circuit 4, generating a differentiated waveform, and the differentiated waveform is transmitted to a comparator 74 in which the negative constant C₃ value, etc., are set, and if the above-described condition 1, etc., are satisfied, then a signal is transmitted to the second arithmetic processing control unit 75 (FPGA), and the second arithmetic processing control unit 75 (FPGA) generates an enable signal.

Namely, it is preferable that a portion from the electrocardiogram waveform input unit 3 to the enable signal generating unit 7 be formed of a hardware circuit. The hardware circuit is a circuit in which signal processing is not performed by software, and thus, signal processing is fast. As a result, a period of time from obtaining of electrocardiogram information to generation of an enable signal can be reduced. Note that signals transmitted from the electrocardiogram waveform input unit 3 to the enable signal generating unit 7 may be analog signals or may be digital signals.

Note that at least one of functions of the electric device for defibrillation 70, e.g., the functions of the electrocardiogram waveform input unit 3, the differentiating circuit 4, the comparator 74, the enable signal generating unit 7, the first arithmetic processing control unit 72, the second arithmetic processing control unit 75, the arithmetic processing control unit 8, the power supply unit 9, and/or the switching unit 10, may be implemented by hardware or may be implemented by software. For details, description of the first embodiment can be referred to.

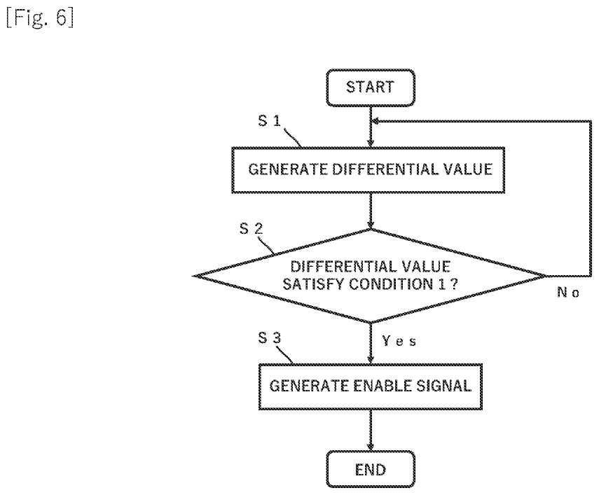

FIG. 6 is a flowchart showing an example of a processing procedure performed by the electric device for defibrillation 70. In an example of FIG. 6, the differentiating circuit 4 generates a differentiated wave value based on electrocardiogram information inputted from the electrocardiogram waveform input unit 3 (step S1). Then, the comparator 74 in which the negative constant C₃ value, etc., are set determines whether the differential value satisfies condition 1 (step S2). If condition 1 is satisfied, then the comparator 74 transmits a signal to the second arithmetic processing control unit 75 (FPGA), and if condition 1 is not satisfied, then the comparator 74 does not transmit a signal to the second arithmetic processing control unit 75 (FPGA). The second arithmetic processing control unit 75 (FPGA) generates an enable signal based on the signal (step S3). In this case, the second arithmetic processing control unit 75 (FPGA) corresponds to the enable signal generating unit 7.

A method for generating a defibrillation signal according to an embodiment of the present invention includes a step of determining whether the following condition 1 is satisfied after the peak 51p of the event 51 estimated to be an R-wave of the electrocardiogram waveform 50 obtained from a human body is surpassed; and a step of generating an enable signal when or after condition 1 is satisfied:

(Condition 1) A differential value generated from the event 51 estimated to be an R-wave is the negative constant C₃ value or less.

It is preferable that the method for generating a defibrillation signal further include a step of determining whether the following condition 2 is satisfied:

(Condition 2) A peak value of a differentiated waveform (hereinafter, simply referred to as “positive wave 61P”) which is a set of differential values generated from a portion of the event 51 estimated to be an R-wave that corresponds to the rise phase 51r occurring before the peak 51p of the event 51 estimated to be an R-wave, is the positive constant C₁ value or more.

It is preferable that the method for generating a defibrillation signal further include a step of determining whether the following condition 3 is satisfied:

(Condition 3) A period of time during which the differential values of the positive wave 61P are the positive constant C₂ value or more is measured and is 10 milliseconds or more and 80 milliseconds or less, the positive constant C₂ value being smaller than the positive constant C₁ value.

It is preferable that the method for generating a defibrillation signal further include a step of determining whether the following condition 4 is satisfied:

(Condition 4) A period of time from when a differential value generated from an event estimated to be an R-wave (hereinafter, simply referred to as “Rₙ₋₁-wave”) immediately before an event estimated to be an R-wave (hereinafter, simply referred to as “Rₙ-wave”) reaches the C₃ value until a differential value generated from the Rₙ-wave reaches the C₃ value is measured and is 50 milliseconds or more.

It is preferable that the method for generating a defibrillation signal include a step of generating an enable signal when or after at least one condition selected from a group consisting of the above-described condition 2, condition 3, and condition 4, and the above-described condition 1 are satisfied.

It is preferable that the method for generating a defibrillation signal include a step of determining whether the following condition 1 is satisfied after the peak 51P of the event 51 estimated to be an R-wave of an electrocardiogram waveform obtained from a human body is surpassed; a step of generating a mark display signal for providing a mark to the event 51 estimated to be an R-wave on the display unit 73 when or after condition 1 is satisfied; and a step of generating an enable signal when or after the step of generating a mark display signal:

(Condition 1) A differential value generated from the event 51 estimated to be an R-wave is the negative constant C₃ value or less.

By the method including the step of generating an enable signal when or after the step of generating a mark display signal, for example, the no-permission mode for defibrillation can be switched to the permission mode after grasping the state of the heart by visually checking R-R intervals, etc., using, as a marker, a mark provided to an event estimated to be an R-wave. By this, defibrillation can be easily performed and safety can be increased.

It is preferable that the method for generating a defibrillation signal include a step of determining whether the following condition 2 is satisfied; and a step of generating a mark display signal for providing a mark to the event 51 estimated to be an R-wave on the display unit 73 when or after condition 2 is satisfied and condition 1 is satisfied:

(Condition 2) A peak value of a differentiated waveform (hereinafter, simply referred to as “positive wave 61P”) which is a set of differential values generated from a portion of the event 51 estimated to be an R-wave that corresponds to a rise phase occurring before the peak 51P of the event 51 estimated to be an R-wave, is the positive constant C₁ value or more.

It is preferable that the method for generating a defibrillation signal include a step of determining whether the following condition 3 is satisfied; and a step of generating a mark display signal for providing a mark to the event 51 estimated to be an R-wave on the display unit 73 when or after condition 2 and condition 3 are satisfied and condition 1 is satisfied:

(Condition 3) A period of time during which the differential values of the positive wave 61P are the positive constant C₂ value or more is measured and is 10 milliseconds or more and 80 milliseconds or less, the positive constant C₂ value being smaller than the C₁ value.

It is preferable that the method for generating a defibrillation signal include a step of generating a mark display signal when or after at least one condition selected from a group consisting of the above-described condition 2 and condition 3, and the above-described condition 1 are satisfied.

It is preferable that the method for generating a defibrillation signal include a step of determining whether the following condition 4 is satisfied; and a step of generating a mark display signal for providing a mark to the event 51 estimated to be an R-wave on the display unit 73 when or after condition 4 is satisfied and condition 1 is satisfied:

(Condition 4) A period of time from when a differential value generated from an event estimated to be an R-wave (hereinafter, simply referred to as “Rₙ₋₁-wave”) immediately before an event estimated to be an R-wave (hereinafter, simply referred to as “Rₙ-wave”) reaches the C₃ value until a differential value generated from the Rₙ-wave reaches the C₃ value is measured and is 50 milliseconds or more.

It is preferable that the method for generating a defibrillation signal include a step of generating a mark display signal when or after at least one condition selected from a group consisting of the above-described condition 2, condition 3, and condition 4, and the above-described condition 1 are satisfied.

The steps of determining whether the above-described conditions 1 to 4 are satisfied can be performed using, for example, a differentiating circuit, an arithmetic processing control unit, a memory, a comparator, a power supply unit, etc., in the electric device for defibrillation 2 or the electric device for defibrillation 70. For details, description of each condition in the electric device for defibrillation 2 or the electric device for defibrillation 70 can be referred to.

For the method for generating a defibrillation signal according to the present invention, the steps do not need to be performed in one electric device for defibrillation and may be performed in different devices.

The present application is a continuation-in-part of International Application No. PCT/JP2021/002247 filed Jan. 22, 2021, which is based upon and claims the benefits of priority to Japanese Application No. 2020-040144 filed Mar. 9, 2020. The entire contents of these applications are incorporated herein by reference.

### DESCRIPTION OF REFERENCE SIGNS

1 defibrillation catheter system
2 electric device for defibrillation
3 electrocardiogram waveform input unit
4 differentiating circuit
5 memory
6 operating unit
7 enable signal generating unit
8 arithmetic processing control unit
9 power supply unit
10 switching unit
10A first switch
10B second switch
11 first connecting unit
12 second connecting unit
13 third connecting unit
14 fourth connecting unit
19 body-surface electrode
20 catheter
21 first electrode group
22 second electrode group
23 third electrode group
25 end tip
26 handle
27 resin tube
31 first conducting wire
32 second conducting wire
33 third conducting wire
34 fourth conducting wire
35 fifth conducting wire
36 sixth conducting wire
37 seventh conducting wire
40 electrocardiograph
50 electrocardiogram waveform
51 event estimated to be an R-wave
51c inflection point of a fall phase of the event estimated to be an R-wave
51d fall phase of the event estimated to be an R-wave
51p peak of the event estimated to be an R-wave
51r rise phase of the event estimated to be an R-wave
52 T-wave
60 differentiated waveform
61 differentiated waveform generated from the event estimated to be an R-wave
61P positive wave
61N negative wave
61b peak of the negative wave
62 differentiated waveform generated from a T-wave
70 electric device for defibrillation
71 A/D converter
72 first arithmetic processing control unit
73 display unit
74 comparator
75 second arithmetic processing control unit
76 mark display signal generating unit

1. An electric device for defibrillation comprising:
an electrocardiogram waveform input unit; and
an enable signal generating unit, wherein
the electric device for defibrillation is configured to generate an enable signal from the enable signal generating unit after a peak of an event is surpassed and when or after condition 1 is satisfied, the event being estimated to be an R-wave of an electrocardiogram waveform, the electrocardiogram waveform being obtained from a human body and inputted from the electrocardiogram waveform input unit, and
the condition 1 is that a differential value in a differentiated waveform generated based on the electrocardiogram waveform, which corresponds to the event estimated to be the R-wave, is a negative constant C₃ value or less.

2. The electric device for defibrillation according to claim 1, wherein
the electric device for defibrillation is configured to generate the enable signal when or after condition 2 is satisfied and the condition 1 is satisfied, and
the condition 2 is that a peak value of the differentiated waveform, which corresponds to a rise phase occurring before the peak of the event estimated as the R-wave of the electrocardiogram waveform, is a positive constant C₁ value or more.

3. The electric device for defibrillation according to claim 2, wherein
the electric device for defibrillation is configured to measure a period of time during which a value of the differentiated waveform, which corresponds to the rise phase occurring before the peak of the event estimated as the R-wave of the electrocardiogram waveform, is a positive constant C₂ value or more, and generate the enable signal when or after the condition 2 and condition 3 are satisfied and the condition 1 is satisfied, and
the condition 3 is that the period of time, during which the value of the differentiated waveform, which corresponds to the rise phase occurring before the peak of the event estimated as the R-wave of the electrocardiogram waveform, is the positive constant C₂ value or more, is 10 milliseconds or more and 80 milliseconds or less, the positive constant C₂ value being smaller than the C₁ value but greater than 0 in the differentiated waveform.

4. The electric device for defibrillation according to claim 1, wherein
the electric device for defibrillation is configured to generate the enable signal when or after condition 4 is satisfied and the condition 1 is satisfied, and
the condition 4 is that a period of time between a moment at which the differential value of Rₙ₋₁-wave reaches the C₃ value and a moment at which the differential value of Rₙ-wave reaches the C₃ value is 50 milliseconds or more, wherein
the Rₙ-wave is the R-wave of the electrocardiogram waveform, and
the Rₙ₋₁-wave is an event estimated to be an R-wave immediately before the Rₙ-wave.

5. The electric device for defibrillation according to claim 1, wherein
a portion from the electrocardiogram waveform input unit to the enable signal generating unit is formed of a hardware circuit.

6. The electric device for defibrillation according to claim 1, comprising a display unit that displays the electrocardiogram waveform, wherein
the electric device for defibrillation is configured to generate a mark display signal for providing a mark to the event estimated to be the R-wave on the display unit from a mark display signal generating unit after the peak of the event estimated to be the R-wave is surpassed and when or after the condition 1 is satisfied.

7. The electric device for defibrillation according to claim 6, wherein
the electric device for defibrillation is configured to generate the mark display signal when or after condition 2 is satisfied and the condition 1 is satisfied, and
the condition 2 is that a peak value of the differentiated waveform, which corresponds to a rise phase occurring before the peak of the event estimated as the R-wave of the electrocardiogram waveform, is a positive constant C₁ value or more.

8. The electric device for defibrillation according to claim 7, wherein
the electric device for defibrillation is configured to measure a period of time during which a value of the differentiated waveform, which corresponds to the rise phase occurring before the peak of the event estimated as the R-wave of the electrocardiogram waveform, is a positive constant C₂ value or more, and generate the mark display signal when or after the condition 2 and condition 3 are satisfied and the condition 1 is satisfied, and
the condition 3 is that a period of time, during which the value of the differential waveform, which corresponds to the rise phase occurring before the peak of the event estimated as the R-wave of the electrocardiogram waveform, is a positive constant C₂ value or more, is 10 milliseconds or more and 80 milliseconds or less, the positive constant C₂ value being smaller than the C₁ value but greater than 0.

9. The electric device for defibrillation according to claim 6, wherein
the electric device for defibrillation is configured to generate the mark display signal when or after condition 4 is satisfied and the condition 1 is satisfied, and
the condition 4 is that a period of time between a moment at which the differential value of Rₙ₋₁-wave reaches the C₃ value and a moment at which the differential value of Rₙ-wave reaches the C₃ value is 50 milliseconds or more, wherein
the Rₙ-wave is the R-wave of the electrocardiogram waveform, and
the Rₙ₋₁-wave is an event estimated to be an R-wave immediately before the Rₙ-wave.

10. A method for generating a defibrillation signal, the method comprising the steps of:
generating a differential value based on an electrocardiogram waveform obtained from a human body,
determining whether condition 1 is satisfied after a peak of an event is surpassed, the event being estimated to be an R-wave of the electrocardiogram waveform; and
generating an enable signal when or after the condition 1 is satisfied, wherein
the condition 1 is that the differential value corresponding to the event estimated to be the R-wave is a negative constant C₃ value or less.

11. The method for generating a defibrillation signal according to claim 10, further comprising the step of determining whether condition 2 is satisfied, wherein
the enable signal is generated when or after the condition 1 and the condition 2 are satisfied, and
the condition 2 is that a peak value of the differentiated waveform, which corresponds to a rise phase occurring before the peak of the event estimated as the R-wave of the electrocardiogram waveform, is a positive constant C₁ value or more.

12. The method for generating a defibrillation signal according to claim 11, further comprising the step of determining whether condition 3 is satisfied, wherein
the enable signal is generated when or after the condition 1, the condition 2 and the condition 3 are satisfied, and
the condition 3 is that a period of time, during which a value of the differentiated waveform, which corresponds to the rise phase occurring before the peak of the event estimated as the R-wave of the electrocardiogram waveform, is a positive constant C₂ value or more, is 10 milliseconds or more and 80 milliseconds or less, the positive constant C₂ value being smaller than the C₁ value but greater than 0 in the differentiated waveform.

13. The method for generating a defibrillation signal according to claim 10, further comprising the step of determining whether condition 4 is satisfied, wherein
the enable signal is generated when or after the condition 1 and the condition 4 are satisfied, and
the condition 4 is that a period of time between a moment at which a differential value of Rₙ₋₁-wave reaches the C₃ value and a moment at which a differential value of Rₙ-wave reaches the C₃ value is 50 milliseconds or more, wherein
the Rₙ-wave is the R-wave of the electrocardiogram waveform, and
the Rₙ₋₁-wave is an event estimated to be an R-wave immediately before the Rₙ-wave.

14. The method for generating a defibrillation signal according to claim 10, comprising the steps of:
determining whether the condition 1 is satisfied after the peak of the event estimated as the R-wave is surpassed;
generating a mark display signal for providing a mark to the event estimated to be the R-wave on a display unit when or after the condition 1 is satisfied; and
generating the enable signal when or after the step of generating the mark display signal.

15. The method for generating a defibrillation signal according to claim 14, comprising the steps of:
determining whether condition 2 is satisfied and generating the mark display signal for providing the mark to the event estimated to be the R-wave on the display unit when or after the condition 2 is satisfied and the condition 1 is satisfied, wherein
the condition 2 is that a peak value of the differentiated waveform, which corresponds to a rise phase occurring before the peak of the event estimated as the R-wave of the electrocardiogram waveform, is a positive constant C₁ value or more.

16. The method for generating a defibrillation signal according to claim 15, comprising the steps of:
determining whether condition 3 is satisfied; and
generating the mark display signal for providing the mark to the event estimated to be the R-wave on the display unit when or after the condition 2 and the condition 3 are satisfied and the condition 1 is satisfied, wherein
the condition 3 is that a period of time, during which a value of the differentiated waveform, which corresponds to the rise phase occurring before the peak of the event estimated as the R-wave of the electrocardiogram waveform, is a positive constant C₂ value or more, is 10 milliseconds or more and 80 milliseconds or less, the positive constant C₂ value being smaller than the C₁ value but greater than 0 in the differentiated waveform.

17. The method for generating a defibrillation signal according to claim 14, comprising the steps of:
determining whether condition 4 is satisfied; and
generating a mark display signal for providing the mark to the event estimated to be the R-wave on the display unit when or after the condition 4 is satisfied and the condition 1 is satisfied, wherein
the condition 4 is that a period of time between a moment at which a differential value of Rₙ₋₁-wave reaches the C₃ value and a moment at which a differential value of Rₙ-wave reaches the C₃ value is 50 milliseconds or more, wherein
the Rₙ-wave is the R-wave of the electrocardiogram waveform, and
the Rₙ₋₁-wave is an event estimated to be an R-wave immediately before the Rₙ-wave.
